(19) United States
(12) Patent Application Publication
(10) Pub. No.: US 2023/0158285 A1
(43) Pub. Date: May 25, 2023

(54) SYSTEM AND METHOD FOR CANNULA FIBER LUMEN STRAIN RELIEF

(71) Applicant: ABIOMED, Inc., Danvers, MA (US)

(72) Inventor: Cam Loughlin, Danvers, MA (US)

(73) Assignee: ABIOMED, Inc., Danvers, MA (US)

(21) Appl. No.: 17/993,076

(22) Filed: Nov. 23, 2022

Related U.S. Application Data
(60) Provisional application No. 63/282,407, filed on Nov. 23, 2021.

(51) Int. Cl. A61M 60/13; A61M 60/174; A61M 60/531; A61M 60/216

(52) U.S. Cl. CPC A61M 60/13; A61M 60/174; A61M 60/531; A61M 60/216; A61M 2205/3306; A61M 25/0054

(57) ABSTRACT

A system that includes a cannula and a flexible hypotube attached to the cannula configured to slidably receive an optical fiber. The system may be configured to have a ratio R1 of a diameter of the outer surface of the hypotube to a diameter of the inner surface of the cannula is 1:5>R1>1:25, and/or where the flexible hypotube comprises a tubular portion containing at least one cut extending from the outer surface at least partially through the sidewall of the flexible hypotube, each cut having a maximum width of between 0.01 and 0.1 mm.

### CROSS-REFERENCE TO RELATED APPLICATIONS

The present application claims priority to U.S. Provisional Patent App. No. 63/282,407, filed Nov. 23, 2021, the contents of which are incorporated by reference herein in their entirety.

### TECHNICAL FIELD

The present disclosure relates to intravascular blood pumps having one or more sensors for measuring, such as for measuring pressures within a patient's vascular system.

### BACKGROUND

Intravascular blood pumps can be introduced into a patient either surgically or percutaneously and used to deliver blood from one location in the bean or circulatory system to another location in the heart or circulatory system. For example, when deployed in the left heart, an intravascular blood pump can pump blood from the left ventricle of the heart into the aorta. Likewise, when deployed in the right heart, an intravascular blood pump can pump blood from the inferior vena cava into the pulmonary artery. Examples of such blood pumps include the Impella® family of devices (Abiomed, Inc., Danvers, Mass.).

Blood pump may have one or more sensors for measuring the patient's vascular system. For example, intravascular blood pumps may include one or more optical sensors for measuring pressures within a patient's vascular system, and particular a patient's ventricular cavity, which may be used for operating the blood pump and/or for assessing the state of health of the patient's heart.

### BRIEF SUMMARY

A first aspect of the present disclosure is drawn to a system including a cannula and a flexible hypotube with specific geometric relationships. The cannula (which may be a flexible flow cannula) has an inner surface and an outer surface, which define a cannula wall having a thickness, the inner surface of the cannula defining a first lumen therethrough. The flexible hypotube is attached to the cannula, and has an outer surface and an inner surface, where the inner surface defines a second lumen therethrough. The system is configured to provide a ratio R1 of a diameter of the outer surface of the hypotube to a diameter of the inner surface of the cannula is 1:5>R1>1:25. The second lumen is arranged to slidably receive an optical fiber therethrough.

Advantageously, the optical fiber is arranged to freely move within the second lumen. In some embodiments, a ratio R2 of a diameter of an outer surface of the optical fiber to a diameter of the inner surface of the flexible hypotube is 1:3>R2>1:1.1

In some embodiments, the system further includes an inflow cage connected at or near a distal portion of the cannula. In some embodiments, a distal portion of the optical fiber is attached to the inflow cage. In some embodiments, the optical fiber includes an optical fiber sensor head at a distal end of the optical fiber, where the sensor may be configured to, e.g., measure ventricular pressure.

In some embodiments, the system further includes a pump operably connected to a proximal portion of the first lumen.

In some embodiments, the flexible hypotube may be positioned within the first lumen. In some embodiments, the flexible hypotube may be positioned outside the first lumen. In some embodiments, at least one portion of the flexible hypotube is positioned within the first lumen and at least one portion of the flexible hypotube is positioned outside the first lumen.

In some embodiments, the flexible hypotube has a distal end, a proximal end, and a tubular portion between the distal end and the proximal end, the tubular portion containing at least one laser cut that extends at least partially through a wall of the flexible hypotube, such as from the outer surface towards the inner surface. In some embodiments, the cuts (or a subset of the cuts) may extend all the way through the wall of the hypotube. In some embodiments, each cut my have a width of between 0.01 mm and 0.1 mm. In some embodiments, the tubular portion may extend all the way between the distal and proximal ends. In such embodiments, the entire hypotube may include cuts other than the distal and proximal ends. In other embodiments, the tubular portion includes a portion of the length of the hypotube between the distal and proximal ends. For example, the tubular portion may be between 20% and 80% of the length of the hypotube. In some embodiments, the tubular portion is centered between the distal and proximal ends. In that regard, the tubular portion may be a central portion of the hypotube.

As will be appreciated, the hypotube and tubular portion may have any suitable cross-sectional shape, although shown as being circular in cross section. For example, in other embodiments, the hypotube and tubular portion may be ovular, triangular, square, other polygonal or other suitable shape.

As described herein, the at least one cut (e.g., laser cut) can exist in a variety of configurations. In some embodiments, the at least one cut defines a helical cut extending axially along the tubular portion of the flexible hypotube.

In some embodiments, the at least one cut comprises a plurality of identical laser cuts. In some embodiments, each of the plurality of identical laser cuts may be offset only axially from all other laser cuts of the plurality of identical laser cuts. In some of these embodiments, each of the plurality of identical laser cuts may be offset axially from all other laser cuts of the plurality of identical laser cuts, and at least one laser cut is offset circumferentially from a neighboring laser cut, such as being offset circumferentially by 45°, 90°, or 180° from a neighboring laser cut. In some of these embodiments, the plurality of identical laser cuts may define at least two helical patterns, such as two helical patterns that are offset circumferentially.

A second aspect of the present disclosure is drawn to a system having a cannula and a flexible hypotube, where the flexible hypotube contains laser cuts in the hypotube. The cannula (which may be a flexible flow cannula) has an inner surface and an outer surface, which define a cannula wall having a thickness, the inner surface of the cannula defining a first lumen therethrough. The flexible hypotube may be attached to the cannula, and has an outer surface and an inner surface, where the inner surface defines a second lumen therethrough. The flexible hypotube includes a tubular portion containing at least one cut extending from the outer surface at least partially through the sidewall of the flexible hypotube, each cut having a maximum width of between 0.01 mm and 0.1 mm. The second lumen is arranged to slidably receive an optical fiber therethrough.

As described herein, the optical fiber may be arranged to freely move within the second lumen of the hypotube. In some embodiments, the system is configured to provide a ratio R2 of a diameter of an outer surface of the optical fiber to a diameter of the inner surface of the flexible hypotube is 1:3>R2>1:1.1. In some embodiments, the system is configured to provide a ratio R1 of a diameter of the outer surface of the hypotube to a diameter of the inner surface of the cannula is 1:5>R1>1:25.

In some embodiments, the flexible hypotube may include a coating or jacket.

In some embodiments, the system also includes an inflow cage connected at or near a distal portion of the cannula. In some embodiments, a distal portion of the optical fiber may be attached to the inflow cage. In some embodiments, the optical fiber includes an optical fiber sensor head at a distal end of the optical fiber, where the sensor may be configured to, e.g., measure ventricular pressure.

In some embodiments, the system further includes a pump operably connected to a proximal portion of the first lumen.

In some embodiments, the flexible hypotube may be positioned within the first lumen. In some embodiments, the flexible hypotube may be positioned outside the first lumen. In some embodiments, at least one portion of the flexible hypotube is positioned within the first lumen and at least one portion of the flexible hypotube is positioned outside the first lumen.

In some embodiments, the flexible hypotube includes a distal portion, a proximal portion, and a tubular portion between the distal portion and the proximal portion, the tubular portion containing at least one cut extending at least partially through the wall of the flexible hypotube, from the outer surface towards the inner surface, each cut having a maximum width of between 0.01 and 0.1 mm. In some embodiments, the cuts may extend all the way through the hypotube.

As with the above, the at least one cut may include a variety of configurations. In some embodiments, the at least one cut defines a helical cut extending axially along the tubular portion of the flexible hypotube. In some embodiments, the at least one cut includes a plurality of identical laser cuts. In some of these embodiments, each of the plurality of identical laser cuts are offset only axially from all other laser cuts of the plurality of identical laser cuts. In some of these embodiments, each of the plurality of identical laser cuts are offset axially from all other laser cuts of the plurality of identical laser cuts, and at least one laser cut is offset circumferentially from a neighboring laser cut, such as being offset circumferentially by 45°, 90°, or 180° from a neighboring laser cut. In some of these embodiments, the plurality of identical laser cuts may define at least two helical patterns, such as two helical patterns that are offset circumferentially.

A third aspect of the present disclosure is drawn to an intravascular blood pump. The pump will generally include a catheter, a pumping device, and at least one sensor. The pumping device may be disposed distally of the catheter and has at its distal end a cannula (which may be a flexible flow cannula) through which blood is either sucked or discharged by the pumping device during operation of the intravascular blood pump. The at least one sensor has at least one optical fiber slidably disposed in a flexible hypotube, the flexible hypotube being at least partially attached to the cannula, the flexible hypotube having a tubular portion containing at least one cut extending from an outer surface of the flexible hypotube towards an inner surface of the flexible hypotube, at least partially through a wall of the flexible hypotube. In some embodiments, the cut(s) may extend all the way through the flexible hypotube wall. In some embodiment, each cut includes a width of between 0.01 mm and 0.1 mm. In some embodiments, the flexible hypotube is configured to minimize and/or prevent breakage of the at least one optical fiber during bending of the flexible hypotube and cannula while the blood pump is guided through a vascular system of a patient.

Advantageously, the optical fiber is arranged to freely move within the second lumen. In some embodiments, the system is configured to provide a ratio R2 of a diameter of an outer surface of the optical fiber to a diameter of the inner surface of the flexible hypotube is 1:3>R2>1:1.1. In some embodiments, the system is configured to provide a ratio R1 of a diameter of the outer surface of the hypotube to a diameter of the inner surface of the cannula is 1:5>R1>1:25.

In some embodiments, the system also includes an inflow cage connected at or near a distal portion of the cannula. In some embodiments, a distal portion of the optical fiber is attached to the inflow cage. In some embodiments, the optical fiber has an optical fiber sensor head at a distal end of the optical fiber, where the sensor may be configured to, e.g., measure ventricular pressure.

In some embodiments, the system further includes a pump operably connected to a proximal portion of the first lumen.

In some embodiments, the flexible hypotube may be positioned within the first lumen. In some embodiments, the flexible hypotube may be positioned outside the first lumen. In some embodiments, at least one portion of the flexible hypotube may be positioned within the first lumen and at least one portion of the flexible hypotube is positioned outside the first lumen.

In some embodiments, the flexible hypotube includes a distal portion, a proximal portion, and a tubular portion having at least one cut extending at least partially through the flexible hypotube (e.g., from the outer surface towards the inner surface). For example, the cut may extend all the way through the hypotube wall. In some embodiments, each cut includes a maximum width of between 0.01 mm and 0.1 mm. The at least one cut may include a variety of configurations. In some embodiments, the at least one cut defines a helical cut extending axially along the tubular portion of the flexible hypotube. In some embodiments, the at least one cut includes a plurality of identical laser cuts. In some of these embodiments, each of the plurality of identical laser cuts are offset only axially from all other laser cuts of the plurality of identical laser cuts. In some of these embodiments, each of the plurality of identical laser cuts are offset axially from all other laser cuts of the plurality of identical laser cuts, and at least one laser cut is offset circumferentially from a neighboring laser cut, such as being offset circumferentially by 45°, 90°, or 180° from a neighboring laser cut. In some of these embodiments, the plurality of identical laser cuts define at least two helical patterns, such as two helical patterns that are offset circumferentially.

A fourth aspect of the present disclosure is a method of reducing strain on an optical fiber during insertion and use of a blood pump. The method includes providing a blood pump having (i) a cannula (which may be a flexible flow cannula) having an inner surface and an outer surface, the inner surface defining a first lumen therethrough, (ii) a flexible hypotube attached to the cannula, the flexible hypotube having an outer surface and an inner surface, the inner surface defining a second lumen therethrough, the flexible hypotube having a tubular portion containing at least one cut extending from the outer surface of the flexible hypotube towards an inner surface of the flexible hypotube at least partially through the flexible hypotube, each opening having a width of between 0.01 and 0.1 mm, and (iii) an optical fiber having an outer surface, the optical fiber laid slidably in the flexible hypotube. The method then includes moving the blood pump through a patient's vascular system, while allowing the optical fiber to move axially within the flexible hypotube while the blood pump is moving.

In some embodiments, the method further includes receiving as an input at an evaluation device a transmitted optical signal from the optical fiber sensor head, and then determining a pressure using the transmitted optical signal.

In some embodiments, the optical fiber is arranged to freely move within the second lumen. In some embodiments, the blood pump of the method is configured to provide a ratio R2 of a diameter of an outer surface of the optical fiber to a diameter of the inner surface of the flexible hypotube is 1:3>R2>1:1.1. In some embodiments, the blood pump of the method is configured to provide a ratio R1 of a diameter of the outer surface of the hypotube to a diameter of the inner surface of the cannula is 1:5>R1>1:25.

In some embodiments, the blood pump of the method further includes an inflow cage connected at or near a distal portion of the cannula. In some embodiments, a distal portion of the optical fiber is attached to the inflow cage. In some embodiments, the optical fiber has an optical fiber sensor head at a distal end of the optical fiber, where the sensor may be configured to, e.g., measure ventricular pressure.

In some embodiments, the blood pump of the method further includes a pump operably connected to a proximal portion of the first lumen.

In some embodiments, the flexible hypotube may be positioned within the first lumen. In some embodiments, the flexible hypotube may be positioned outside the first lumen. In some embodiments, at least one portion of the flexible hypotube is positioned within the first lumen and at least one portion of the flexible hypotube is positioned outside the first lumen.

In some embodiments, the flexible hypotube has a distal portion, a proximal portion, and a tubular portion between the distal portion and the proximal portion, the tubular portion containing at least one cut at least partially through the flexible hypotube extending from the outer surface towards the inner surface, each cut having a width of between 0.01 and 0.1 mm.

As with the above, the at least one cut may include a variety of configurations. In some embodiments, the at least one cut defines a helical cut extending axially along the tubular portion of the flexible hypotube. In some embodiments, the at least one cut includes a plurality of identical laser cuts. In some of these embodiments, each of the plurality of identical laser cuts are offset only axially from all other laser cuts of the plurality of identical laser cuts. In some of these embodiments, each of the plurality of identical laser cuts are offset axially from all other laser cuts of the plurality of identical laser cuts, and at least one laser cut is offset circumferentially from a neighboring laser cut, such as being offset circumferentially by 45°, 90°, or 180° from a neighboring laser cut opening. In some of these embodiments, the plurality of identical laser cuts define at least two helical patterns, such as two helical patterns that are offset circumferentially.

### BRIEF DESCRIPTION OF DRAWINGS

FIG. 1 is a depiction of a blood pump laid through the aorta, extending through the aortic valve into the left ventricle and having an integrated pressure sensor.

FIG. 2A is a cross-section schematic representation of an embodiment of a flexible hypotube.

FIG. 2B is a cross-section schematic representation of an embodiment of a cannula, flexible hypotube, and optical fiber.

FIG. 2C is a cross-section schematic representation of an embodiment of a cannula where the flexible hypotube is disposed inside the cannula.

FIG. 3A is a schematic representation of a flexible hypotube with cuts as disclosed.

FIGS. 3B-3G are depictions of side views of alternate examples of cuts made in a flexible hypotube.

FIG. 3H is an axial cross-sectional view of a portion of a flexible hypotube coupled to a cannula.

FIG. 3I is a magnified or enlarged image of a flexible hypotube coupled to a cannula.

FIG. 4 is a depiction of a pumping device of the blood pump from FIG. 1 in greater detail.

FIG. 5 is a flowchart of a disclosed method.

### DETAILED DESCRIPTION

As is known, blood pumps may have sensors for monitoring a patient. For example, intravascular blood pumps may include optical sensors, such as for measuring pressures within a patient's vascular system, and particular a patient's ventricular cavity, which may be used for operating the blood pump and/or for assessing the state of health of the patient's heart.

As appreciated by the inventors, the cannula, on its way to placement in the heart, may be subjected to great bends or flexions which can exert non-negligible tensile and compressive stresses on an optical fiber laid along the cannula, which may cause damage to the optical fiber. For example, in an illustrative example, the optical fiber may become detached from the pump housing during insertion due to tensile and compressive stresses. This also may apply to optical fibers made of glass. Although such optical fibers are normally covered with a thin plastic coating, such as polyimide (Kapton), which offers some protection from breakage, the danger of breakage during insertion due to tensile and compressive stresses could still be problematic. For example, such damage may result in the entire blood pump having to be replaced (e.g., if the optical fiber breaks).

Accordingly, the inventors have recognized the advantages of a system and method for relieving the strain of an optical fiber associated with a cannula, such as a flexible flow cannula. As described herein, in some embodiments the system may include a flexible hypotube within which an optical fiber is slidably disposed. In this regard, the hypotube may form a protective sleeve within which the optical fiber may be seated. In some embodiments, the hypotube is associated with a cannula. For example, the hypotube may be disposed inside or outside the cannula. The hypotube may include one or more cuts (e.g., laser cuts).

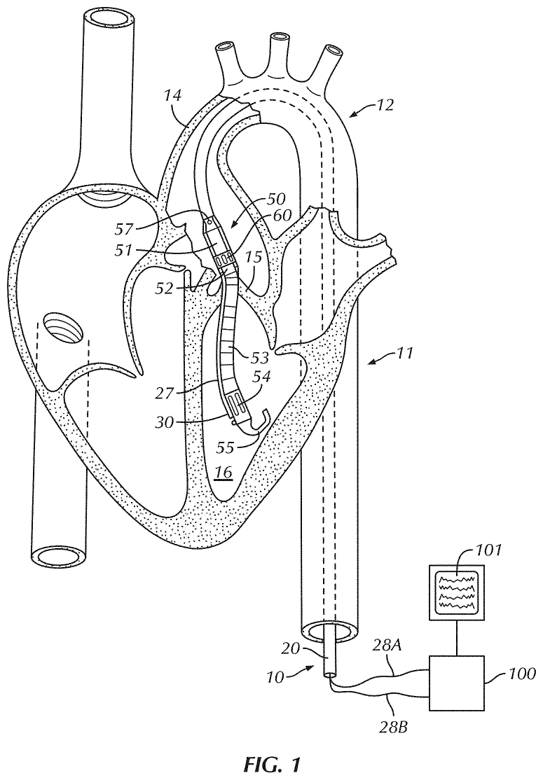

Turning now to the figures, FIG. 1 shows an exemplary intravascular blood pump having a catheter 10 which may be introduced into a patient's heart. For example, as show in this figure, the pump may be inserted into the descending aorta 11 retrograde in some embodiments. As is known, the descending aorta is part of the aorta 12 which first ascends from the heart and then descends and has the aortic arch 14. At the beginning of the aorta 12 there is located the aortic valve 15 which connects the left ventricle 16 to the aorta 12 and through which the intravascular blood pump may extend. As will be appreciated, blood pumps may be inserted into other suitable portions of the

[symbol]

As shown in FIG. 1, the intravascular blood pump may include a rotary pumping device 50 fastened at the distal end of the catheter hose 20 and having a motor section 51 and a pump section 52 (sometimes referred to herein as simply the “pump”) disposed at an axial distance therefrom, as well as a cannula 53 (which may be a flexible flow cannula) protruding in the distal direction from the inflow end of the pump section 52 and having a suction inlet 54 located at its end. Distally of the suction inlet 54 there is provided a soft-flexible tip 55, which can be configured for example as a “pigtail” or in a J shape. Through the catheter hose 20 there may extend different lines and devices which may be important for operating the pumping device 50.

In some embodiments, the pump may include two optical fibers 28A, 28B that may be attached at their proximal end to an evaluation device 100. These optical fibers 28A, 28B may be respectively part of an optical sensor (such as a pressure sensor) whose sensor heads 30 and 60 may be located in the vicinity of the suction inlet 54, on the one hand, and on the outside on the housing of the pump section 52, on the other hand. FIG. 1 shows the sensor head 30 as being on the outside of the blood pump, but the sensor head could also be located internal to the blood pump, preferably within an inflow cage, the inflow cage having lumens therethrough that define the suction inlet 54. In such embodiments, information (e.g., pressure information) may be transmitted by the sensor heads 30 and 60 to the evaluation device 100 and may be converted into electrical signals in the evaluation device 100 and displayed e.g., on a display screen 101.

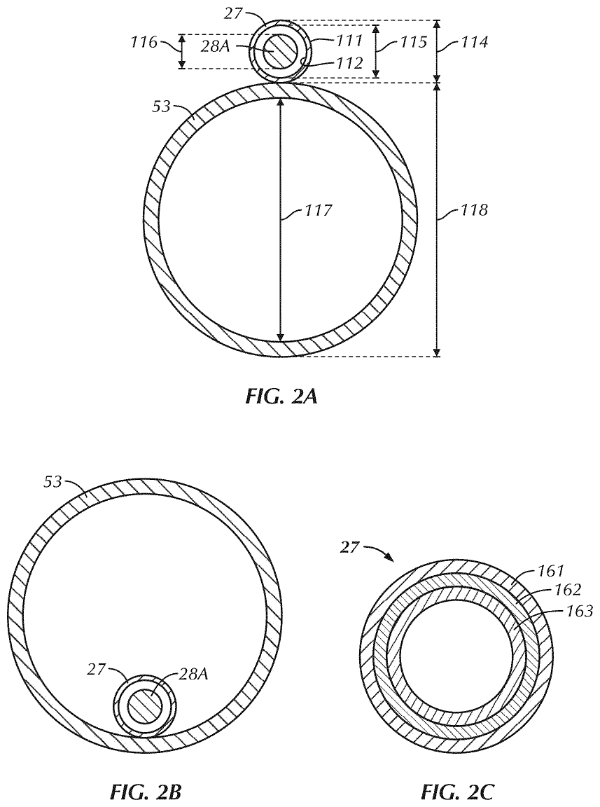

According to an aspect of the present disclosure, and as shown in FIGS. 2A and 2B, the intravascular pump may include a flexible hypotube 27 for receiving an optical fiber. For example, the optical fiber may be slidably received in the flexible hypotube. As will be appreciated in view of these figures, the hypotube may be extend along an inner or outer surface of the cannula. As will be further appreciated, in some embodiments, the hypotube may extend partially along the inside and partially along the outside surfaces of the cannula. For example, in some embodiments, one or more portions of the flexible hypotube may be disposed on the inside of the cannula, while one or more other portions of the flexible hypotube may be disposed on the outside of the cannula. One or more portions of the flexible hypotube also may extend on a portion of the blood pump other than the cannula (e.g., on or in the inflow cage).

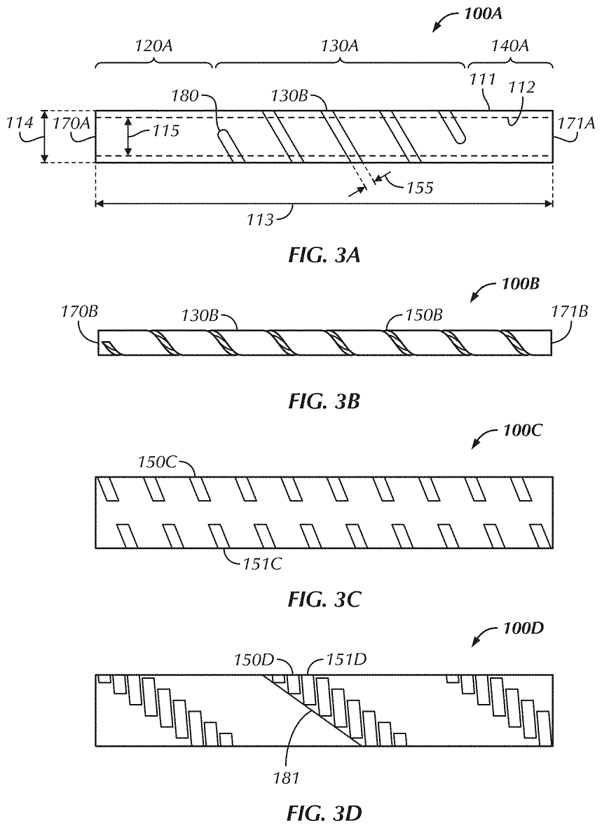

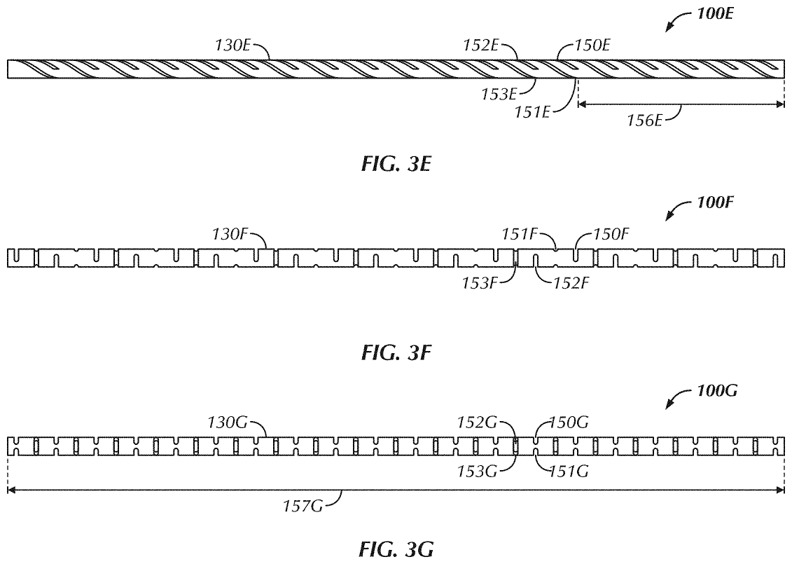

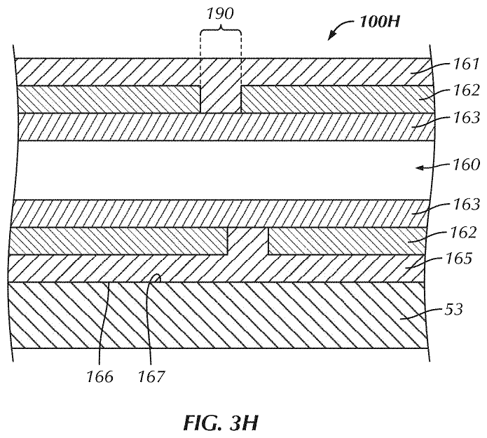

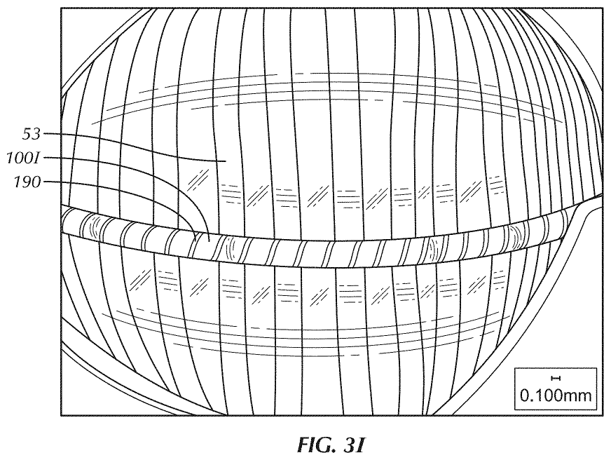

In some embodiments, the flexible hypotube may have a distal end, a proximal end, and a tubular portion including one or more cuts as described herein. As described herein, the tubular portion may extend along a length of the flexible hypotube, between the proximal and distal ends. In some embodiments, the tubular portion may extend along an entire length of the hypotube. In other embodiments, the tubular portion may extend along only a portion of the length of the hypotube. For example, in some embodiments, the tubular portion may include a middle portion of the hypotube. In such embodiments, the hypotube may include a distal portion, a middle portion, and the proximal portion. See, e.g., FIG. 3A, in which the flexible hypotube 100A includes a proximal portion 120A, a middle portion 130A, and a distal portion 140A.

As shown in FIGS. 2A and 3A, for example, flexible hypotube may be attached to the flexible flow cannula. The flexible hypotube may have an outer surface 111 and an inner surface 112, where the inner surface defines a lumen extending from a proximal end 170A to a distal end 171A. In some embodiments, the hypotube may extend along a portion of the outer surface of the flow cannula (see, e.g., FIG. 2A). In other embodiments, the hypotube may extend along a portion of the inner surface of the flow cannula (see, e.g., FIG. 2B). In still other embodiments, the hypotube may extend along a portion of each of the inner and outer surfaces of the flow cannula.

The flexible hypotube 27 in which the optical fibers 28A, 28B are laid can, in some embodiments, extend shortly (e.g., less than 6 inches) into the catheter hose 20, but can also extend completely through the catheter hose 20 (see FIG. 1) and end in a corresponding plug at the end of the line for insertion of the relevant pressure sensor into a connection of the evaluation device 100.

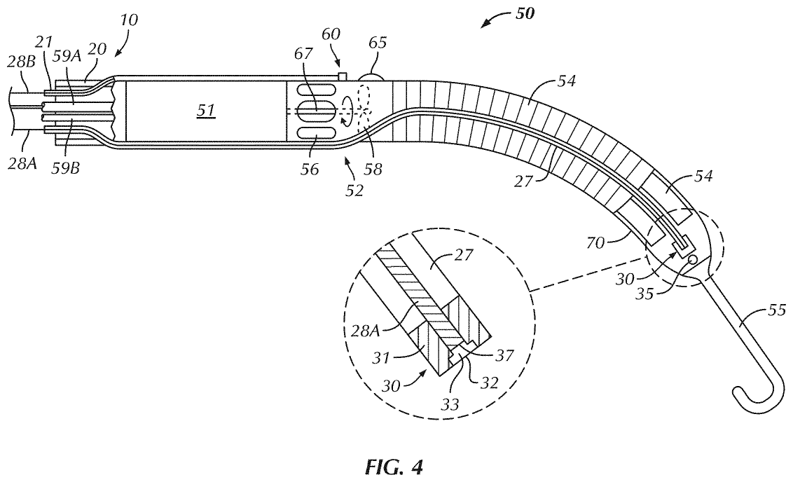

In some embodiments (see, e.g., FIG. 4), the cannula 53 may have a precurvature which may facilitate the laying of the device in the patient's vascular system. Due to this precurvature, the cannula 53 may have an inner radius of curvature and an outer radius of curvature between which, substantially in the middle, a neutral bending plane extends. In such embodiments, the hypotoube may be configured to bend with the precurvature of the flow cannula.

The flexible hypotube may include a single walled, hollow tube. In some embodiments, the hypotube may include one or more coatings around a single walled, hollow tube. For example, as seen in FIG. 2C, in some embodiments, the hypotube 27 may comprise an outer jacket 161 external to a single walled, hollow tube 162, the single-walled hollow tube having one or more cuts described herein. In some embodiments, the hypotube 27 may comprise an inner coating 163 internal to a single walled, hollow tube 162. In some embodiments, the hypotube 27 may comprise both an outer jacket 161 and an inner coating 163. As will be appreciated, in some embodiments, the flexible hypotube may include only an inner jacket or only an outer jacket. In some embodiments, the outer jacket and inner coating are comprised of the same material. In some embodiments, the outer jacket and inner coating are comprised of different materials.

The flexible hypotube will have a length 113. In some embodiments, the hypotube may be between 4 and 8 cm in length, such as between 5 and 7 cm in length.

As shown in FIGS. 2A and 3A, the flexible hypotube may have an outer surface 111 defining an outer diameter 114 from a proximal end to the distal end. The flexible hypotube also may include an inner surface 112 having an inner diameter 115 that defines a lumen therethrough (e.g., from the proximal end 170 to the distal end 171). In some embodiments, the flexible hypotube may have an inner diameter between 0.130 mm and 0.275 mm, such as between 0.140 mm and 0.160 mm. The difference between the outer diameter 114 and the inner diameter 115 may define a wall thickness of the flexible hypotube. In some embodiments, the wall thickness may be between 0.045 mm and 0.090 mm, such as between 0.050 mm and 0.070 mm.

Referring briefly to FIGS. 2A and 2B, an optical fiber 28A may be present within a lumen of a flexible hypotube 27, which is attached to the cannula 53. For example, in some embodiments, the hypotube may be attached to an outer surface of the hypotube (see FIG. 2A), while in other embodiments, the hypotube may be attached to an inner surface of the cannula (see FIG. 2B). Accordingly, in FIG. 2A, the flexible hypotube and optical fiber may be positioned outside the first lumen, while in FIG. 2B, the flexible hypotube and optical fiber may be positioned within the first lumen. As will be appreciated, a first portion of the flexible hypotube may be positioned within the first lumen while a portion of the flexible hypotube is positioned the first lumen.

As represented in these views, an outer diameter 114 of the flexible hypotube 27 may be smaller than the inner diameter 117 which defines a lumen through the cannula 53. In some embodiments, these components may be configured such that a ratio R1 of a diameter 114 of the outer surface of the hypotube 27 to a diameter 117 of the inner surface of the cannula 53 is 1:5>R1>1:25. That is, that the inner diameter of the cannula may be between 5 and 25 times larger than the outer diameter of the hypotube, such as between 15 and 25 times larger than the outer diameter of the hypotube (that is, 1:15>R1>1:25).

Further, the inner diameter 115 of the flexible hypotube 27 may be larger than the outer diameter 116 of the optical fiber 28A. In some embodiments, these components are configured such that a ratio R2 of a diameter 116 of an outer surface of the optical fiber 28A to a diameter 115 of the inner surface of the flexible hypotube 27 may be 1:3>R2>1:1.1, such as 1:2>R2>1:1.1.

In some embodiments, the optical fiber may be arranged to freely move within the second lumen, such that the optical fiber can move, axially, independent of movement of the flexible hypotube. In some embodiments, this freedom may be a result of the optical fiber not being connected to the flexible hypotube. In some embodiments, this freedom may be a result of there being slack, or excess, optical fiber within the flexible hypotube. For example, in some embodiments, the optical fiber may be attached to the hypotube (i.e., adhered to the hypotube, or otherwise restricted from moving axially independently of the hypotube) at or near the proximal end of the hypotube, while a linear length of optical fiber within the flexible hypotube may be longer (e.g., up to 1.1-1.2 times longer) that the linear length of the flexible hypotube, such that when the cannula and flexible hypotube bend, there may be sufficient excess optical fiber within the hypotube that the optical fiber does not experience substantial tension. As will be appreciated, the optical fiber also may be attached to other suitable portions of the hypotube and/or to the pump (e.g., at an inflow cage).

As shown in FIGS. 3A-3G, the flexible hypotube may include a tubular portion with at least one cut. As described herein, the cut(s) may be configured to control the ease (or resistance) the hypotube exhibits when bending in certain directions. In some embodiments, the cuts may be arranged such that the hypotube may bend in only one direction, such as in a prescribed bend direction of the cannula on or within which the hypotube is placed. The hypotube also may be configured to bend in all directions.

As seen in FIG. 3A, in some embodiments of the system, the flexible hypotube may include a distal portion 140A at or near a distal end 171, a proximal portion 120A at or near a proximal end 170, and a tubular portion 130A having at least one cut 150A extending at least partially through the flexible hypotube from the outer surface 111 towards the inner surface 112 (e.g., only partially through the wall of the flexible hypotube). In some embodiments, the distal portion may extend a distance from a distal end and the proximal portion may extend a distance from the proximal end. In some embodiments, each cut may extend completely through the wall of the flexible hypotube (e.g., from the outer surface through to the inner surface).

While the length of each cut may vary, each cut may have a width w 155 of between 0.01 and 0.1 mm. For purposes herein, a cut made between point A and point B along the surface of the hypotube may follow a pathline that is a straight, a curved, or a freeform path. The pathline may have a distance (i.e., the length of the pathline), and the means of forming the cut (such as a laser cut) may define the width w of the cut. In some embodiments, the width w of a cut may be constant along the entire pathline. In other embodiments, the width w of a cut may vary. For example, in some embodiments, the width of a cut may be constant except for the very ends 180 of each cut (which may be rounded, etc.). In other embodiments, the width of the cut may vary in other suitable portions

As shown in FIGS. 3A-3G, different patterns of cuts may be utilized.

As seen in FIGS. 3A and 3B, in some embodiments, the at least one cut 150A, 150B comprises a single cut that defines a helical (or spiral) cut extending axially along a cut length 130A, 130B of the flexible hypotube 100A, 100B. The spiral pattern may have a constant pitch or may have a varying pitch. In some embodiments, the pitch may be between 0.3 mm and 1.0 mm. In one embodiment, the pitch may be 0.5 mm.

As will be appreciated, the length of the cut (e.g., the length of the tubular portion) may extend along an entire length of the hypotube (e.g., in FIG. 3B, between the distal end 171B and proximal end 170B of the hypotube 100B). In other embodiments, such as those depicted in FIG. 3A, the length of the cut (e.g., a length of the tubular portion) may include a central portion of the hypotube. For purposes herein, the central portion 130A may include between 20% and 80% of a length of the hypotube. In some embodiments, the central portion is centered between the distal end 171A and proximal end 170A of the hypotube.

While some embodiments utilize only a single cut, in some embodiments, the at least one cut may comprise a plurality of identical cuts, such as a plurality of identical laser cut openings.

For example, as shown in FIGS. 3C and 3D, the cut may include partial spiral cuts. As will be appreciated, the plurality of cuts may extend around the circumferences of the hypotube and along a length of the hypotube. As seen in FIG. 3C, in some embodiments, a first partial spiral cut 150C and a second partial spiral cut 151C may be configured such that if the pathline of the first spiral cut and the pathline of the second spiral cut were extended, they would intersect and form a continuous spiral pattern. As seen in FIG. 3D, in some embodiments, the pathlines of a first partial spiral cut 150D and a second partial spiral cut 151D may be parallel to each other but not intersect if extended. Rather, the partial cuts may be configured such that a portion of each cut (such as a bottom edge, a top edge, a center point, etc.), if connected by an imaginary line 181, would define a spiral pattern. In some embodiments, each cut may be configured such that a pathline of the cut is linear. In some embodiments, each cut may be configured such that a pathline of the cut is non-linear. In some embodiments, each cut may be made such that the pathline of the cut is in a plane normal to the axis of the hypotube. In some embodiments, each cut may, independently, be made such that the pathline of the cut is in a plane that is not normal to the axis of the hypotube.

As seen in FIG. 3E, the at least one cut may include a repeating pattern of interrupted helical or spiral patterns or cuts, including first spiral cut 150E, an identical second spiral cut 151E, an identical third spiral cut 152E, and an identical fourth spiral cut 153E. The spiral pattern(s) may have a constant pitch or may have a varying pitch. In some embodiments, the pitch may be between 0.3 mm and 1.0 mm. For example, the pitch may be 0.5 mm.

As shown in FIG. 3E, the first cut 150E and second cut 151E may start at the same axial distance 156E from a distal end of the flexible hypotube, but may be offset circumferentially from each other (that is, the end of each cut is the same axial distance from the distal end of the hypotube, but the patterns are “rotated” around the axis of the hypotube in relation to each other). In some embodiments, the circumferential offset is ±45°, ±90°, or 180° from a neighboring laser cut opening. Neighboring laser cut opening can generally be considered as two different laser cut openings where there is no intervening other laser cut opening between them. In FIG. 3E, the first cut 150E and the third cut 152E may be only offset axially from each other, not circumferentially, while the first cut 150E and fourth cut 153E may be offset circumferentially and axially from each other. As such, in FIG. 3E, at least one cut of the plurality of cuts (e.g., third cut 152E) may be offset only axially from at least one other cut (e.g., first cut 150E), at least one other cut (e.g., second cut 151E) of the plurality of cuts may be offset only circumferentially from the at least one other cut, and at least one additional cut (e.g., fourth cut 153E) of the plurality of cuts is offset both axially and circumferentially from the at least one other cut.

While FIG. 3E shows interrupted spirals, where each spiral or helical pattern or cut (150E, 151E, 152E, 153E) may extend only a portion of the length of the tubular portion 130C, in other embodiments, the plurality of identical laser cuts can define at least two helical patterns, that each extends substantially the entire length of the tubular portion of the hypotube, or substantially the entire length of the hypotube. For example, in some cases, two or more helical patterns may be cut into the hypotube, where the patterns are only offset circumferentially.

Combinations of these cuts are also envisioned in other embodiments. For example, in some embodiments, one first long spiral cut may extend substantially the entire length of the tubular portion. In another embodiment, the hypotube may include a plurality of additional interrupted spiral cuts that are each offset circumferentially and/or axially from the first long spiral cut.

As seen in FIG. 3F, in some embodiments, the flexible hypotube 100F may include a tubular portion with at least one cut having a repeating pattern of cuts (150F, 151F, 152F, 153F), where each cut is made orthogonal to the axis of the flexible hypotube (e.g., the cut is made only in a circumferential direction). In such embodiment, each cut may include first cut 150F, an identical second cut 151F, an identical third cut 152F, and an identical fourth cut 153F. As seen in FIG. 3F, each of the plurality of identical laser cut openings may be offset axially from all other laser cut openings of the plurality of identical laser cut openings. Further, as seen in FIG. 3F, at least one laser cut opening may be offset circumferentially from a neighboring laser cut opening. For example, in FIG. 3F, 151F is offset from both neighboring laser cut openings, 150F and 152F. In FIG. 3F, each laser cut opening may be offset axially by a small amount and circumferentially by 90 degrees from a previous laser cut opening in the pattern. That is, after 150F is cut, the hypotube can be rotated 90 degrees, optionally moved axially a small amount, and identical second cut 151F can be made.

In some embodiments, the at least one laser cut opening may be offset circumferentially by ±45°, ±90°, or 180° from a neighboring laser cut opening.

In some embodiments, at least one laser cut opening may be only offset circumferentially from a neighboring laser cut, and at least one laser cut opening may be offset circumferentially and axially from a neighboring laser cut.

As seen in FIG. 3G, in some embodiments, the flexible hypotube 100G may include a cut length 157G (as discussed previously, this cut length may be some or all of the entire length of the hypotube) with at least one cut comprises a repeating pattern of cuts (150G, 151G, 152G, 153G), where at least one laser cut opening (151G) is only offset circumferentially from a neighboring laser cut (150G), and at least one laser cut opening (152G, 153G) is offset circumferentially and axially from the neighboring laser cut (150G). In FIG. 3G, the repeating pattern consists of two pairs of cuts (150G and 151G, 152G and 153G), where each cut in a pair are the same axial distance along the hypotube (i.e., they are not offset axially from each other), but are offset circumferentially 180 degrees from each other. For example, first cut 150G may be circumferentially offset 180 degrees from second cut 151G. The second pair of cuts may be offset axially from the first pair of cuts and offset circumferentially 90 degrees. That is, a third cut 152G may be offset axially by a small amount and offset circumferentially 90 degrees from first cut 150G, and fourth cut 153G may be offset axially by a small amount and offset circumferentially −90 degrees (or offset +270 degrees) from first cut 150G.

As seen in FIGS. 3H and 3I, in some embodiments, the gaps 190 formed by the cuts in the hypotube 100H, 100I may be filled in, such as with a polymer. For example, in some embodiments, the gaps 190 may be filled with a urethane material, such as polyurethane (e.g., a thermoformed polyurethane). In some embodiments, the gaps may be filled by coating the hypotube with the polymer material. In some embodiments, the hypotube may include a polymer jacket. In such embodiments, the hypotube may have a smooth inner and/or outer surface. As seen in FIG. 3H, in some embodiments, a surface 166 of the hypotube 100H may be coupled to a surface 167 of a cannula 53. The hypotube 100I may comprise an inner coating 163, such as a low-friction coating (which may be, e.g., polytetrafluoroethylene (PTFE)) or a polyurethane, that defines the lumen 160 therethrough. The gaps 190 may be filled in, such as with a polymer layer 161. In some embodiments, the polymer layer may not only form an outer layer of the hypotube 100H and may also form an outer coating or layer of the cannula 53. In some embodiments, the outer layer of the hypotube may be separate from the outer layer of the cannula, and thus the outer layer of the hypotube may be in contact with an outer layer of the cannula 53. In some embodiments, the outer layer 161 of the hypotube may be comprised of the same material forming an outer coating or layer 165 of the cannula 53. In some embodiments, the outer layer 161 of the hypotube may be comprised of a different material forming an outer coating or layer 165 of the cannula 53.

Referring to FIG. 4, a pump may be operably connected to a proximal portion of the first lumen of cannula 53 (which may be a flexible flow cannula). The cannula preferably has a suction inlet 54 located at its distal end. In preferred embodiment, the suction inlet 54 may comprise a plurality of apertures formed on an inflow cage 70 that is coupled to the cannula 53. In some implementations, the inflow cage may be comprised of stainless steel.

In some embodiments, a distal portion of the optical fiber is attached to the inflow cage 70, and in some embodiments, attached to an internal surface of the inflow cage.

Referring to FIGS. 1 and 4, also disclosed is an intravascular blood pump. The blood pump may comprise a catheter, a pumping device, and at least one sensor.

The pumping device 50 may be disposed distally of the catheter 10 and may have at its distal end, a cannula 53 (which may be a flexible flow cannula) through which blood either sucked or discharged by the pumping device 50 during operation of the intravascular blood pump. The at least one sensor having at least one optical fiber 28A may be laid slidably in a flexible hypotube 27, the flexible hypotube being at least partially attached to the cannula (e.g., on an inner or outer surface of the cannula. As described herein, the flexible hypotube may include a tubular portion containing at least one cut extending from an outer surface of the flexible hypotube towards an inner surface of the flexible hypotube at least partially through the flexible hypotube, each cut having a width of between 0.01 and 0.1 mm. In some embodiments, the flexible hypotube may be configured to minimize or prevent breakage of the at least one optical fiber during bending of the flexible hypotube and/or cannula, and/or to minimize or prevent detachment of an optical sensor from the pump (e.g., an optical sensor attached to an inlet housing) while the blood pump is guided through a vascular system (e.g., 11, 12, 14, 15, 16) of a patient.

The components of the blood pump, including the cannula, flexible hypotube, and sensor (optical fiber, sensor head, etc.) may be configured as described in any of the previous embodiments.

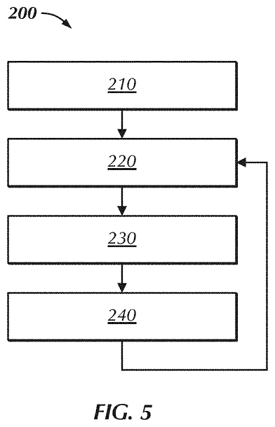

Also disclosed is a method of reducing strain on an optical fiber during insertion and use of a blood pump. Referring to FIG. 5, the method 200 generally comprises providing 210 a blood pump with an optical fiber as described above. For example, in some embodiments, the blood pump may comprise (i) a cannula having an inner surface and an outer surface, the inner surface defining a first lumen therethrough, (ii) a flexible hypotube attached to the cannula (e.g., on the inner and/or outer surface), the flexible hypotube having an outer surface and an inner surface, the inner surface defining a second lumen therethrough, the flexible hypotube having a tubular portion containing at least one cut extending from the outer surface of the flexible hypotube towards an inner surface of the flexible hypotube at least partially through the wall of the flexible hypotube, each opening having a width (e.g., maximum width) of between 0.01 and 0.1 mm, and (iii) an optical fiber having an outer surface, the optical fiber laid slidably in the flexible hypotube. In other embodiments, the blood pump may comprise (i) a cannula having an inner surface and an outer surface, the inner and outer surface defining a cannula wall having a thickness, the inner surface of the cannula defining a first lumen therethrough, and (ii) a flexible hypotube attached to the cannula, the flexible hypotube having an outer surface and an inner surface, the inner surface of the flexible hypotube defining a second lumen therethrough. In some embodiments, the second lumen may be arranged to slidably receive an optical fiber therethrough, with the cannula and flexible hypotube being configured such that a ratio R1 of a diameter of the outer surface of the hypotube to a diameter of the inner surface of the cannula may be 1:5>R1>1:25.

After the blood pump has been provided, the blood pump can then be moved through a patient's vascular system. Rather than keeping the optical fiber from moving, the optical fiber may be allowed 220 to move axially within the flexible hypotube while the blood pump is moving.

After the blood pump is moved, an evaluation device (c.f. FIG. 1, device 100) may receive 230 as an input an optical signal transmitted from an optical fiber sensor head, through the optical fiber. The evaluation device can then use that received input in order to determine 240 a pressure and/or signal-to-noise ratio (SNR).

In some embodiments, the process of moving the blood pump and measuring pressures is repeated at least once. In some embodiments, it is repeated until the determined pressure indicates the blood pump is positioned correctly.

Referring again to FIG. 4, in some embodiments, the glass membrane 32 in the sensor head may be pressure-sensitive and be deformed in response to the amount of pressure acting on the sensor head 60 (or 30). For example, the deformation of the glass membrane 32 may cause the light to be reflected and coupled back into the optical fiber 28A. Optical fiber 28A transmits the optical signal from the optical fiber sensor to an evaluation device. For example, as mentioned above, the optical signals transmitted by the sensor heads 30 and 60 can be converted into electrical signals in the evaluation device 100 and displayed, e.g., on a display screen 101.

As seen in FIG. 4, the optical fiber 28A the pressure-measuring catheter may have a sensor head 30 having a head housing 31 which contains a thin glass membrane 32 which terminates a cavity 33. The glass membrane 32 may be pressure-sensitive and be deformed in dependence on the size of a pressure acting on the sensor head 30. Through the reflection on the membrane the light exiting from the optical fiber 28A may be reflected modulatingly and coupled back into the optical fiber. The coupling can be effected either directly into the optical fiber 28A or indirectly via a bottom 37 terminating the cavity 33 in a vacuum-tight manner. In some embodiments, the bottom 37 may be an integral part of the head housing 31. Thus, the specification of the pressure in the cavity 33 can be effected independently of the mounting of the optical fiber 28A. At the proximal end of the optical fiber 28A, i.e., in the evaluation device 100, there may be located a digital camera, such as e.g., a CCD camera or a CMOS, which evaluates the incoming light in the form of an interference pattern. In dependence thereon, a pressure-dependent electrical signal may be generated. The evaluation of the optical image or optical pattern delivered by the camera and the computation of the pressure may be affected through the evaluation unit 100. The latter passes the already linearized pressure values to the control means, which also controls the power supply to the motor-operated pumping device 50 in dependence on the effected evaluation of the pressure signal.

In some embodiments, the evaluation device 100 may alternatively, or additionally, calculate a signal-to-noise ratio (SNR) based on the transmitted optical signal. For example, the optical signal that is transmitted from the distal sensor head 60 to the evaluation device 100 using optical fiber 28A can be used by the evaluation device 100 to calculate the SNR of the optical signal. The SNR can be linked to the mechanical vibrations of a pumping device 50. When the pumping device 50 is stopped, the motor current is zero and the mechanical vibrations of the pumping device 50 are at a minimum. During this state, the SNR may be relatively large because the noise level of the optical signal is low. When the pumping device 50 is running, the motor current is greater than zero and the mechanical vibration of the pumping device 50 increases. During this state, the SNR may be relatively low because the noise level of the optical signal is large.

The pumping device 50 from FIG. 1 is represented in further detail in FIG. 4. As seen in FIG. 4, a drive shaft 57 may protrude from the motor section 51 into the pump section 52, which drives an impeller 58 by means of which, during operation of the blood pump, blood may be sucked through the blood pass-through openings 54 at the distal end of the cannula 53 and discharged proximally of the impeller 58 through the blood-flow pass-through openings 56. The pumping device 50 can also pump in the reverse direction when it is adapted accordingly. Leading through the catheter hose 20 of the catheter 10 to the pumping device 50 may be the above-mentioned optical fibers 28A, 28B, on the one hand, and a power-supply line 59A for the motor section 51 and a purge-fluid line 59B.

Instead of the optical pressure sensor described with reference to FIG. 4, which may work on, e.g., the Fabry-Pérot principle, other optical sensors (including other optical pressure sensors) with one or more optical fibers can also be employed. For example, in some embodiments, the optical sensor may be configured to measure a ventricular pressure.

The components of the blood pump, including the cannula, flexible hypotube, and sensor (optical fiber, sensor head, etc.) may be configured as described in any of the previous embodiments.

Those skilled in the art will recognize or be able to ascertain using no more than routine experimentation many equivalents to the specific embodiments of the invention described herein. Such equivalents are intended to be encompassed by the following claims.

1. A system comprising:
a cannula having an inner surface and an outer surface, the inner and outer surface defining a cannula wall having a thickness, the inner surface of the cannula defining a first lumen therethrough;
a flexible hypotube attached to the cannula, the flexible hypotube having an outer surface and an inner surface, the inner surface of the flexible hypotube defining a second lumen therethrough, wherein the second lumen is arranged to slidably receive an optical fiber therethrough;
wherein a ratio R1 of a diameter of the outer surface of the hypotube to a diameter of the inner surface of the cannula is 1:5>R1>1:25.

2. The system according to claim 1, wherein the optical fiber is arranged to freely move within the second lumen.

3. The system according to claim 2, wherein a ratio R2 of a diameter of an outer surface of the optical fiber to a diameter of the inner surface of the flexible hypotube is 1:3>R2>1:1.1.

4. The system according to claim 1, wherein the cannula is a flexible flow cannula.

5. The system according to claim 1, further comprising an inflow cage connected at or near a distal portion of the cannula.

6. The system according to claim 5, wherein a distal portion of the optical fiber is attached to the inflow cage.

7. The system according to claim 6, wherein the optical fiber comprises an optical fiber sensor head at a distal end of the optical fiber.

8. The system according to claim 7, wherein the optical fiber sensor is configured to measure ventricular pressure.

9. The system according to claim 1, further comprising a pump operably connected to a proximal portion of the first lumen.

10. The system according to claim 1, wherein the flexible hypotube is positioned within the first lumen.

11. The system according to claim 1, wherein the flexible hypotube is positioned outside the first lumen.

12. The system according to claim 1, and at least a second portion of the flexible hypotube is positioned outside the first lumen.

13. The system according to claim 12, wherein the second portion extends into an inflow cage of a pumping device.

14. The system according to claim 1, wherein the flexible hypotube has a distal end, a proximal end, and a tubular portion having one or more cuts that extend at least partially from an outer surface of the hypotube to an inner surface of the hypotube.

15. The system according to claim 14, wherein the tubular portion includes at least one cut extending completely through the flexible hypotube and extending from the outer surface towards the inner surface, optionally wherein each cut has a width of between 0.01 and 0.1 mm.

16. The system according to claim 14, wherein the tubular portion is a distal portion.

17. The system according to claim 14, wherein the flexible hypotube has a proximal portion, a distal portion, and a middle portion between the proximal portion and distal portion.

18. The system according to claim 17, wherein the at least one cut defines a helical cut extending axially along the middle portion of the flexible hypotube.

19. The system according to claim 17, wherein the at least one cut comprises a plurality of identical laser cuts.

20. The system according to claim 19, wherein each of the plurality of identical laser cuts are offset only axially from all other laser cuts of the plurality of identical laser cuts.

21. The system according to claim 19, wherein each of the plurality of identical laser cuts are offset axially from all other laser cut openings of the plurality of identical laser cuts, and at least one laser cut is offset circumferentially from a neighboring laser cut.

22. The system according to claim 21, wherein at least one laser cut is offset circumferentially by 45°, 90°, or 180° from a neighboring laser cut.

23. The system according to claim 19, wherein the plurality of identical laser cuts define at least two helical patterns.

24. A system comprising:
a cannula having an inner surface and an outer surface, the inner surface of the cannula defining a first lumen therethrough;
a flexible hypotube attached to the cannula, the flexible hypotube having an outer surface and an inner surface, the inner surface and outer surface defining a sidewall therebetween, the inner surface of the flexible hypotube defining a second lumen therethrough, wherein the second lumen is arranged to slidably receive an optical fiber therethrough, the flexible hypotube;
wherein the flexible hypotube comprises a tubular portion containing at least one cut extending from the outer surface at least partially through the sidewall of the flexible hypotube, each cut having a width of between 0.01 and 0.1 mm.

25-45. (canceled)

46. An intravascular blood pump, comprising:
a catheter;
a pumping device disposed distally of the catheter and having at its distal end a cannula through which blood is either sucked or discharged by the pumping device during operation of the intravascular blood pump; and
at least one sensor having at least one optical fiber laid slidably in a flexible hypotube, the flexible hypotube being at least partially attached to the cannula, the flexible hypotube comprising a tubular portion having at least one cut extending from an outer surface of the flexible hypotube towards an inner surface of the flexible hypotube and at least partially through the flexible hypotube, each cut having a width of between 0.01 and 0.1 mm.

47-83. (canceled)
